(19) United States
(12) Patent Application Publication
(10) Pub. No.: US 2011/0190701 A1
(43) Pub. Date: Aug. 4, 2011

(54) METHODS, SYSTEMS AND DEVICES FOR RECORDING DATA

(75) Inventors: Axel Remde, Luetzelfueh (CH); Gilbert Schiltges, Kirchberg (CH)

(73) Assignee: ROCHE DIAGNOSTICS OPERATIONS, INC., Indianapolis, IN (US)

(21) Appl. No.: 12/854,498

(22) Filed: Aug. 11, 2010

(30) Foreign Application Priority Data
Aug. 12, 2009 (EP) 09010374.8

(51) Int. Cl. A61M 5/142; G06Q 50/00; G06F 11/07; G10L 15/26; A61B 5/00

(52) U.S. Cl. 604/131; 705/3; 714/48; 704/235; 600/365; 714/E11.025; 704/E15.043

(57) ABSTRACT

Methods, systems and devices for recording data are described herein. In one embodiment a method for recording data for keeping a diary may include: storing primary data indicative of a measured blood glucose level and/or an administrated dose of insulin in a memory of an electronic diary device; receiving a spoken voice message with a microphone, wherein the spoken voice message is indicative of supplementary data directly related to the primary data; and storing the supplementary data in the memory of the electronic diary device, wherein the primary data is stored with a primary time stamp and the supplementary data is stored with the primary data, the primary time stamp or a supplementary time stamp.

### CROSS REFERENCE TO RELATED APPLICATIONS

The present application claims priority to European Patent Application No. EP09010374 filed Aug. 12, 2009, which is herein incorporated by reference in its entirety.

### TECHNICAL FIELD

The embodiments described herein relate to methods, devices and systems for recording data and keeping a diary of a long-term testing of the glucose levels in the blood of a patient and/or of an insulin therapy of a patient suffering from diabetes.

### BACKGROUND

A diary may be kept to supplement long-term medical testing of the glucose levels in the blood of a person and diabetes therapy with insulin. Generally, the person undergoing the testing or the therapy may record all relevant events and data such as, for example, all events and data concerning blood glucose measurements, insulin administrations, meal intakes, sportive activities, illnesses, potential malfunctions of the therapy devices and the like. While there are known devices which automatically record some of the events and data such as glucose measurements and insulin administrations, further valuable information is not recorded in a diary.

Hand-written diaries may be utilized to record information. As an alternative, several electronic systems are known, such as the ACCU-CHEK® Pocket Compass software of Roche Diagnostics AG, Switzerland, which runs on a standard Personal Digital Assistant.

Devices for administering medicaments and for testing blood glucose levels are known in the art and allow recording supplementary data (e.g. the patient's lifestyle, and therapy related data) in a memory of the device.

EP 2 060 284 A1 discloses a device for the self-administration of liquid drugs in adjustable doses. The device includes a dosing module for administering the liquid drug and an electronics module with a memory. Data related to the dosed amount of the liquid drug and therapy can be stored together in the memory with a time stamp. The dosed amount of liquid drug is automatically stored when the user administers the drug. Data related to therapy can be entered manually into the device.

EP 1 369 688 A2 discloses a device for testing the glucose levels in the blood of persons. While the measured glucose values are automatically stored in the device, supplementary data such as food consumption, medication intake and physical exercise must be entered manually.

In practice, diaries recorded with either of the above methods and devices often are incomplete and/or include errors. Many persons do not keep a diary because they feel that diary-keeping is cumbersome. It is estimated by medical doctors that typically 40% of all diary entries may be missing, incomplete or wrong.

Therapy diaries, however, may assist health care professionals for monitoring the therapy and for long-term optimization. In some cases, diaries may be required by law in order to prove therapy quality (in many countries, for example, for car drivers).

### SUMMARY

According to one embodiment, a method for recording data for keeping a diary may include: storing primary data indicative of a measured blood glucose level and/or an administrated dose of insulin in a memory of an electronic diary device; receiving a spoken voice message with a microphone, wherein the spoken voice message is indicative of supplementary data directly related to the primary data; and storing the supplementary data in the memory of the electronic diary device, wherein the primary data is stored with a primary time stamp and the supplementary data is stored along with the primary data and the primary time stamp or is stored separately with a supplementary time stamp.

In another embodiment, a medical device for keeping a diary of a long-term testing of glucose levels in blood of a person and/or of an insulin therapy of the person is disclosed. The medical device may include an electronics module comprising a memory which stores primary data with a primary time stamp and supplementary data either along with the primary data and the primary time stamp or separately with a supplementary time stamp, the primary data indicative of a measured blood glucose level and/or an administrated dose of insulin and the supplementary data being directly related to the measured blood glucose level and/or the administrated dose of insulin. The medical device may include also a clock circuitry which generates the primary time stamp and the supplementary time stamp, and a voice recording unit which receives a spoken voice message, wherein the voice message is indicative of the supplementary data.

In a further embodiment, a system may include a medical device and an external device. The external device downloads data from the medical device via a wired or wireless interface. The medical device includes a memory which stores primary data together with a primary time stamp and supplementary data together with the primary time stamp or a supplementary time stamp. The primary data may be indicative of a measured blood glucose level and/or an administrated dose of insulin and the supplementary data may be directly related to the measured blood glucose level and/or the administrated dose of insulin. The medical device may further include a clock circuitry which generates the primary time stamp and the supplementary time stamp and a voice recording unit which receives a spoken voice message, wherein the voice message is indicative of the supplementary data.

It is to be understood that both the foregoing general description and the following detailed description describe various embodiments and are intended to provide an overview or framework for understanding the nature and character of the claimed subject matter. The accompanying drawings are included to provide a further understanding of the various embodiments, and are incorporated into and constitute a part of this specification. The drawings illustrate the various embodiments described herein, and together with the description serve to explain the principles and operations of the claimed subject matter.

### BRIEF DESCRIPTION OF THE DRAWINGS

FIG. 1 schematically depicts a front view of a medical device according to one or more embodiments shown and described herein;

FIG. 2 schematically depicts a simplified structural diagram of the medical device of FIG. 1 according to one or more embodiments shown and described herein;

FIG. 3 schematically depicts a front view of a second embodiment of a medical device according to one or more embodiments shown and described herein;

FIG. 4 schematically depicts a simplified structural diagram of the medical device of FIG. 3 according to one or more embodiments shown and described herein;

FIG. 5 schematically depicts a display indication of a medical device displaying a table-like data diary according to one or more embodiments shown and described herein; and

FIG. 6 schematically depicts a structural diagram of a medical diary system according to one or more embodiments shown and described herein.

### DETAILED DESCRIPTION

Methods, devices and systems that record data for diary keeping are described herein. The embodiments described herein may improve the quality of medical testing and therapy, for example in the field of diabetes treatment. The embodiments described herein may also improve the quality of therapy diaries and may reduce the effort required of a person undergoing testing or therapy to keep a therapy diary.

According to one embodiment, a method for recording data for diary keeping includes recording long-term testing data of glucose levels in a person's blood and/or recording data related to insulin therapy of a person suffering from diabetes. Primary data representing the measured blood glucose levels and/or the administrated doses of insulin and supplementary data are stored with a primary time stamp in a memory of an electronic diary device. The supplementary data may be directly related to the measured blood glucose levels and/or administrated doses of insulin such as, for example, information about the reason for a bolus administration of insulin or a comment to a measured blood glucose level. The supplementary data may be stored together with supplementary time stamps or together with the primary time stamp, i.e., the time stamp of the related measured blood glucose level and/or administrated dose of insulin. When the supplementary data are stored together with supplementary time stamps, the supplementary data may be linked to the related data such that they can be later combined for presentation and evaluation by pointers, corresponding identifiers, and the like. The primary time stamp or the supplementary time stamp may include the date and the time of entry of the instance of data into the diary device or the date and the time of storing of the instance of data in the memory of the diary device. It is to be appreciated that the above mentioned link between the supplementary data and the related primary data may be an explicit link (e.g., a pointer(s), a corresponding identifier(s), etc.), or vice versa. Alternatively, the link may be implicit via the time stamp information. For example, it may be assumed that the supplementary data (e.g., a voice message and/or data relating to food intake in general, physical activities, illnesses or ingestion of further medicaments, etc.), is directly related to primary data (e.g., a blood glucose value and/or an administered dose of insulin), if the time stamp for the primary data and the time stamp for the supplemental data deviate by no more than a predetermined time period, e.g., 1 or 2 minutes.

In another embodiment, the supplementary data can be entered into a diary device by speaking voice messages into a microphone of the diary device and stored in a memory of the diary device. For example, supplementary data may be spoken into and stored in the diary device immediately upon their occurrence. Therefore, supplementary data may be recorded according to the embodiments described herein in a convenient, fast and discreet manner without interfering with other activities. The relative ease of diary keeping via the embodiments described herein may increase the amount of supplementary data recorded. Such an increase in supplementary data recording may improve the accuracy by reducing the number of incorrectly remembered or forgotten data entries.

In yet another embodiment, the method includes storing additional supplementary data together with additional time stamps in a memory of an electronic diary device. The additional supplementary data is not directly related to the measured blood glucose levels and/or an administrated dose of insulin such as, for example, data relating to food intake in general, physical activities, illnesses or ingestion of further medicaments. The memory in which these data are stored may be the same memory in which the data directly related to the measured blood glucose levels and/or an administrated dose of insulin are stored in or a separate memory. The additional time stamp may include the date and the time of entry of the instance of data into the diary device or the date and the time of storing of the instance of data in the memory of the diary device. In additional embodiments, the additional supplementary data can be entered into the diary device by speaking a voice message into a microphone of the device.

In a further embodiment, the method includes storing supplementary data such as voice messages entered into the diary device in a memory together with a time stamp and playing back the supplementary data from the memory for checking and/or analysis.

In still a further embodiment of the present disclosure, the method includes transforming a voice message into text automatically with a diary device, and storing the text together with a time stamp in a memory. The voice message may be stored in the memory in addition to the text, or the voice message may be discarded after the transformation. The text resulting from the transformation of the voice message may be utilized for later analysis such as automated analyses of the data stored in the memory.

In an embodiment of the method, according to the embodiments described herein, voice messages entered into the diary device comprise key words and/or numeric values. Once a key word and/or numeric value is identified by the diary device, the key word and/or numeric value is transformed into text and stored as text together with a time stamp in the memory of the diary device. Alternatively, a key word and/or numeric value may be stored in memory as a code. For example, key words may be coded and stored in the device in form of a list. The list may comprise key words arranged in order with a number rather than the full text transformed from the voice message. Storing only the identified keywords and/or numeric values of a voice message as text or code together with a time stamp may facilitate automated analyses of the stored data and may reduce the memory requirements of the diary device.

In another embodiment, the method includes confirming the text generated by the diary device through transformation of a voice message before it is stored in the memory of the device. The text may include keywords and/or numeric values identified in the voice message. The text, keywords, numeric values, or a combination thereof may be checked and confirmed by a user to reduce data errors efficiently.

In a further embodiment, the method includes receiving a voice command with the diary device and executing an action according to the voice command. For example, a user may control diary keeping of the diary device through the user's voice. The diary device may be operably connected with or comprise means for testing the glucose level in the blood of a patient. Therefore, in one embodiment, upon testing the glucose level and generating a test result, the test result is automatically stored in the memory of the diary device together with a time stamp. Additionally, a voice message related to the test result and/or a transcription into text of such a voice message may be stored in the memory. The voice message or text may be stored with the related test result or with a separate time stamp independent of the time stamp of the test result.

Furthermore, the diary device may be operably connected with or comprise means for administration of insulin such as, for example, an insulin pump or an insulin pen. In one embodiment, upon an administration of insulin, data related to the dose of insulin administered is automatically stored in a memory of the diary device together with a time stamp. In another embodiment, an insulin pump administers doses according to a scheduled administration profile (“basal” administration profile) and unscheduled administration (“bolus” administration). Since the basal administration is known, only the bolus administration (e.g., a quantitative value of the bolus dose) is stored in memory for diary keeping. Additionally, a voice message related to the dose and/or a transcription into text of such a voice message may be stored in the memory. The voice message or text may be stored with the related dose or with a separate time stamp independent of the time stamp of the related dose.

According to the embodiments described herein, the diary device may control the means for testing the glucose level and/or the means for administration of insulin. In one embodiment, a specific amount of insulin to be administered is calculated by the diary device and the administration thereof is initiated by the diary device. The calculation may be based upon for example, supplementary data, time of day, a result of testing the glucose level, or combinations thereof. Additionally, it is noted that such calculations may be based upon data entered into the diary via a voice message. In another embodiment, a voice message and/or a transcription of such a voice message into text related to the diary device controlled insulin administration is stored in the memory. The voice message or text may be stored with data related to the diary device controlled insulin administration or a time stamp independent of the diary device controlled insulin administration.

In one embodiment, the method comprises storing malfunction information related to malfunctions or defects in the memory of the diary device together with time stamps. The malfunction information may be related to malfunctions or defects of the diary device and/or of connected or integral devices such as, for example, connected means for testing the glucose level in the blood of a patient, connected means for administration of insulin, and the like. Additionally, explanatory information may be entered as an explanatory voice message into the diary device. The explanatory voice message and/or a transcription of the explanatory voice message into an explanatory text may be stored in memory. The explanatory voice message or explanatory text may be stored with information related to the malfunction or defect or an explanatory time stamp independent of the malfunction or defect (e.g., a time stamp indicative of when the explanatory voice message was recorded). The stored explanatory voice message, explanatory text, malfunction information related to malfunctions or defects, or combinations thereof may then be utilized for analysis of data stored in memory of the diary device.

In another embodiment, the method comprises downloading data stored in the diary device to an external device such as a personal computer, and analyzing the data with the external device. It is noted that, some of the data may have been entered into the device by a spoken voice message. For example, the diary device may be a portable device that is small and simple relative to a stationary computer and the stationary computer may be utilized to perform extensive analyses of the data and visualization of the results of the analyses.

Medical devices that record data for diary keeping are described herein. The medical devices may record primary data, i.e. long-term testing data of the glucose levels in the blood of a patient and/or an insulin therapy data of a patient suffering from diabetes. In one embodiment, the medical device comprises a memory for storing, together with a primary time stamp, data representing measured blood glucose levels and/or representing administrated doses of insulin (e.g., bolus insulin administrations), and supplementary data. The supplementary data may be directly related to the measured blood glucose levels and/or administrated doses of insulin such as, for example, information about the reason for a bolus administration of insulin or a comment to a measured blood glucose level. The supplementary data may be stored in memory together with a supplementary time stamp. The supplementary time stamp may include the date and the time of entry of the data into the medical device or the date and the time of storing the data into the memory.

In another embodiment, the medical device further comprises a clock circuitry for generating the time stamps, and a voice recording unit for entering the supplementary data. For example, the supplementary data may be a spoken voice message which is stored in the memory of the medical device. Supplementary data (e.g., data related to a measured blood glucose level and/or an administered dose of insulin) may be spoken into the medical device immediately upon their occurrence (e.g., the time of measurement and/or administration). The immediate recording of data may reduce the occurrence of incorrectly remembered or completely forgotten data.

In a further embodiment, the medical device comprises a memory for storing additional supplementary together with additional time stamps. The additional supplementary data is data not directly related to the measured blood glucose levels and/or an administrated dose of insulin such as, for example, data related to food consumption, physical activities, illnesses and intake of further medicaments of the patient. In that case, the medical device may furthermore comprise a voice recording unit for entering at least a part of the additional supplementary data which are to be stored in a memory into the medical device by speaking of voice messages. Additionally, the medical device may be configured for one-handed operation when entering voice messages.

In yet a further embodiment, the medical device is configured to store voice messages entered via the voice recording unit together with time stamps. Voice messages stored with time may be played back for later checking and converted into a text transcription.

In one embodiment, the medical device comprises a playback unit such as a loudspeaker or an equivalent. The playback unit may be used for playing back the voice messages, or for providing indications relating to the order in which information should be entered into the medical device. The playback unit may also be used as general indication device for reminder alerts, indicating device errors and the like.

According to the embodiments described herein, the medical device may comprise a text recognition unit for voice-to-text transformation of an entered voice message. Once the voice message is converted into text, the text may be stored in the memory with time stamps. In one embodiment, the transformation takes place automatically when a voice message has been entered to facilitate analysis of data stored in memory. Additionally, the text recognition unit may be adapted to identify and transform into text only certain keywords and/or numeric values contained in an entered voice message.

The medical device may be configured to require confirmation of a text conversion prior to storing the text in memory. In one embodiment, the medical device comprises a display and one or more control buttons. For example, the text resulting from the speech-to-text transformation performed by the text recognition unit may be displayed on the display for rechecking by the user. The text on the display may be confirmed by a user by pushing one or several of the control buttons for confirmation. Once confirmed, the text may then be stored together with a time stamp in the memory. Confirmation of text may be utilized to reduce errors resulting from an erroneous transformation.

In a further embodiment, the text recognition unit is configured to learn new keywords. For example, a user may train the text recognition unit to learn a new keyword by playing back a voice message stored in the memory and entering a corresponding text via an input unit of the medical device. Accordingly, the medical device may be trained to the requirements and language of the user.

In still a further embodiment, the medical device is controlled by a received voice command. For example, a user may speak voice commands to the medical device to facilitate diary keeping.

According to the embodiments described herein, the medical device may further comprise a data interface for downloading data stored in the memory of the medical device to an external device such as a personal computer and its equivalents for analyzing the data. The medical device may be a portable device that is small and simple relative to the external device, which may be stationary. Once the data is downloaded, the external device may be utilized to perform analysis of the data and visualization of the results of the analysis.

Medical devices that record data for diary keeping are described herein. In one embodiment, the medical device comprises an electronics module, means for testing the glucose level in the blood of a patient and/or means for administration of insulin such as, for example, an insulin pen or an insulin pump, or any other injection module for insulin. The electronics module is linked together with the means for testing the glucose level and/or with the means for administration of insulin. When the electronics module is linked with the means for testing the glucose level, the test result can be stored in the memory of the electronics module together with a primary time stamp upon a testing of the glucose level. Similarly, when the electronics module is linked with means for administration of insulin the dose of insulin can be stored in the memory of the medical device together with a primary time stamp upon an administration of insulin. Additionally, a voice message related to the test result or the insulin dose may be received by the medical device and transcribed into text. The voice message and/or the text may be stored in a memory of the medical device with a supplementary time stamp, a primary time stamp or with the related test result or insulin dose.

In another embodiment of the medical device, the storage of data takes place automatically in order to reduce the probability that data related to the testing or to the therapy will be lost.

In a further embodiment of the medical device, the electronics module, the means for testing the glucose level in the blood of a patient and/or the means for administration of insulin are contained in separate housings and are linked with each others by interfaces such as, a wireless interface.

In one embodiment, the medical device comprises means for testing the glucose level in the blood of a patient, means for the computation of a dose of insulin, and means for administration of insulin such as an injection module for insulin. The computation of the dose of insulin to be administered may be based upon supplementary data and/or based on the result of testing the glucose level stored in a memory of a medical device. For example, the computation of the insulin dose may be partially based on stored data which have been entered as voice messages.

The medical device may be configured to be controlled from a housing containing the electronics module to facilitate testing and/or therapy. For example, when the medical device comprises means for administration of insulin, the means for administration of insulin may be controlled from a housing containing the electronics module. Similarly, when the medical device comprises means for testing the glucose level in the blood of a patient, the means for testing the glucose level in the blood of a patient may be controlled from a housing containing the electronics module.

In an embodiment of the present disclosure, the medical device comprises an electronics module that stores malfunction information about malfunctions and/or defects to reduce misinterpretations of the data stored in the memory of the medical device during subsequent analysis. The malfunctions and/or defects may be from the electronics module, the means for testing the glucose level in the blood of a patient, the means for administration of insulin, or combinations thereof. The malfunction information may be stored in memory together with malfunction time stamps. In another embodiment, explanatory information related to the malfunction information is entered as an explanatory voice message. The explanatory voice message may be transformed into an explanatory text. The explanatory voice message or explanatory text may be stored with the malfunction information, the malfunction time stamp or an explanatory time stamp independent of the information about malfunctions and/or defects in the memory 13, dose memory 16, or voice memory 18 of the medical device.

Systems that record data for medical diary keeping are described herein. The system comprises a medical device, as described herein. The system further comprises an external unit for storing data downloaded from a memory and for analyzing the downloaded data. The data may be downloaded by a wired or wireless interface. For example, the medical device may be portable (small and simple relative to the external unit) and the external unit such as a stationary computer may be utilized to perform extensive analysis of the data and visualization of the results of the analysis.

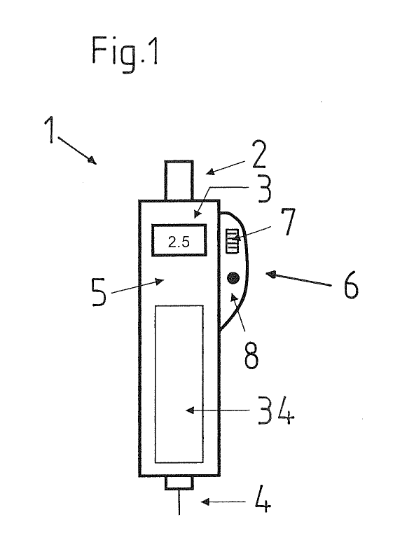

FIG. 1 schematically depicts a medical device 1 such as, for example, an insulin pen according to the embodiments described herein. In one embodiment, the medical device 1 comprises a dose setting knob 2, a dose scale 3, a cannula 4, and a housing 5. For example, a dose of insulin may be injected by actuating an unlocking button (not shown) on the housing 5 to transition the dose setting knob 2 from a retracted position (not shown) inside the housing 5 to the a projecting position. The dose amount of insulin is set by rotating the dose setting knob 2 and is indicated on the dose scale 3 (e.g., a mechanical or electronic digital scale). The insulin dose may be administered from an insulin cartridge (not shown) inside the housing 5 via the cannula 4 such as a detachable single-use cannula by manually pressing the dose setting knob 2 to transition from the projecting position to the retracted position. The filling level of the cartridge is visible through a cartridge window 34 arranged at the front of the housing 5.

The medical device 1 may further comprise a voice recording unit 6 for recording voice notes via a microphone 7.

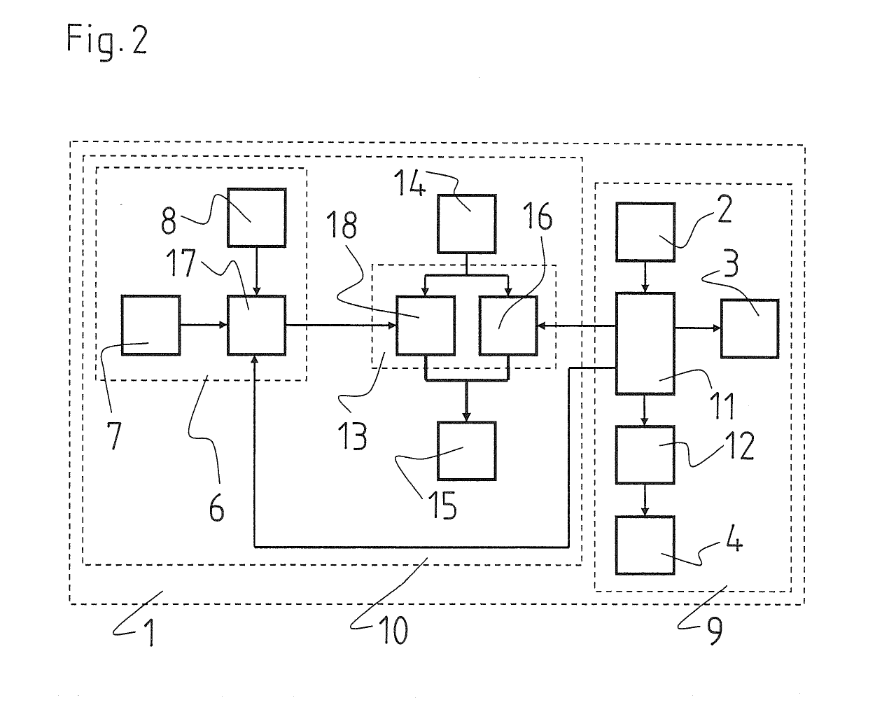

Referring collectively to FIGS. 1 and 2, further embodiments of the medical device 1 will be described in greater detail. In one embodiment, the medical device 1 comprises an injection module 9 and an electronics module 10. The injection module 9 comprises a dose setting knob 2, a dosing mechanism 11, a drug cartridge 12, a cannula 4, and a dose scale 3. The dose scale 3 may be a mechanical digital display as depicted in FIG. 1, an analogue indicator such as, for example, a micrometer screw, or an electronic dose display. In some embodiments, the electronics module 10 may comprise the dose scale 3, for example when the dose scale is an electronic dose display. In the embodiments described herein, the injection unit may comprise reusable and/or disposable components.

In one embodiment, the electronics module 10 comprises a memory 13, a clock circuitry 14, a data interface 15, and a voice recording unit 6. When a drug dose is administered, the corresponding dose amount is stored in a dose memory 16 along with a dose time stamp generated by the clock circuitry 14. For this purpose, the dosing mechanism 11 comprises an electronic encoder for transforming rotations of the dose setting knob 2 indicative of a dose amount into an electrical signal indicative of the dose amount. The dose amount is stored in the dose memory 16 after the administration of the dose amount. For example, after the dose setting knob 2 is transitioned from the projecting position to the retracted position which indicates a drug administration is complete.

In addition to storing the dose amount in the dose memory 16 when the drug administration is complete, the voice recording unit 6 may be automatically activated. The voice recording unit 6 comprises a microphone 7 and a processing unit 17. For example, when activated, the microphone 7 generates an electrical signal upon receiving a spoken voice message. The electrical signal may then be amplified, digitized and processed by the processing unit 17. The processing unit 17 comprises components (e.g., a microprocessor) and implements functionality (e.g., software) of devices commonly called digital dictating devices. The digitized voice message is stored in a voice memory 18 along with a supplementary time stamp which is generated by the clock circuitry 14 or together with the dose time stamp of the administered drug dose it relates to. In another embodiment, the voice recording unit 6 implements a voice controlled activation (VCA) such as, for example VCA from dictating devices, such that recording takes place only if and as long as the voice recording unit 6 receives a spoken message. Once the spoken message is received, the voice memory 18 may store the voice message in a memory-efficient format which is suited for storing voice recordings such as, for example, the “dds” format or another suited format for voice recording.

For example, the voice recording unit 6 may be automatically activated upon completion of a drug injection to allow a person with diabetes to add voice messages with supplementary information for every administration without requiring additional handling steps. Additionally, recording may be started and stopped at any time via a manual recording button 8 in order to enter voice messages. Such voice messages may be stored in the voice memory 18 together with a supplementary time stamp.

According to an embodiment described herein, the medical device 1 is designed as a durable device and comprises the electronics module 10 and the voice recording unit 6 both of which are disposed within a housing 5. For example, the durable device may comprise reusable components and disposable components such as, a drug cartridge 12 that is replaceable and cannula 4 configured for a single use. Alternatively, the injection module 9 may be designed for single use and the electronics module 10 may be designed as a durable unit which is releasably attached to the injection module 9.

For diary-keeping purposes, the data stored in both the dose memory 16 and the voice memory 18 may be downloaded to an external device via a data interface 15. The data interface 15 may be a wireless RF interface such as a Bluetooth interface, an IR interface or a wired interface such as a USB interface. The external device may be a personal computer (PC) which runs corresponding diary software or a dedicated diabetes management device similar to a personal digital assistant (PDA). The data interface 15 may also upload parameters to the medical device 1 such as, for example, the date and time of day.

In an alternative embodiment, the medical device 1 comprises a playback unit for playing back of voice messages stored in the voice memory 18.

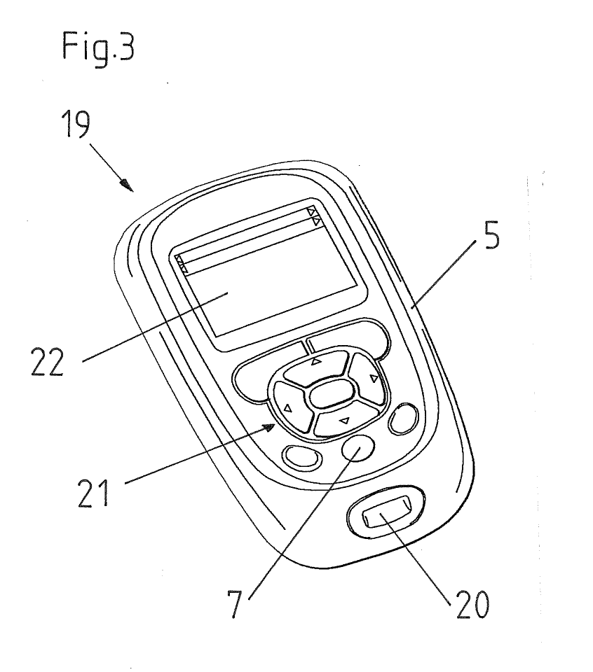

Referring now to FIG. 3, a diabetes management device 19 is schematically depicted. In one embodiment, the diabetes management device 19 integrates the functions of a blood glucose meter, a remote controller for an insulin pump, and an electronic diabetes diary with data presentation and analysis functionality. The diabetes management device 19 comprises a test strip socket 20 for receiving a blood glucose test strip such as, for example blood test strips available from Roche Diagnostics AG, Switzerland, under the name ACCU-CHEK® Aviva. In another embodiment, the diabetes management device 19 comprises a number of test strips that are used one after another (e.g., in a disposable drum or on a flexible tape) such as the devices available from Roche Diagnostics AG, Switzerland, under the names ACCU-CHEK® Compact plus or ACCU-CHEK® Mobile.

The diabetes management device 19 comprises a keyboard 21 that operates the diabetes management device 19 and a display 22 that shows results. For example, an insulin pump for insulin therapy can be remotely controlled via the keyboard 21 and with insulin therapy results shown on the display 22 as well. In one embodiment, the diabetes management device 19 comprises a pump interface 26 (not shown) such as a RF interface or another suited wireless technology. Blood glucose measurements, insulin administration as well as further therapy-relevant information, such as insulin pump errors, are stored in the diabetes management device 19 for diary-keeping purposes. The display 22 may be a graphical display that allows presentation of the data in an appropriate form for analysis and interpretation such as curves of blood glucose values over time, time scales indicating insulin administrations, several types of statistical analysis, and the like. In another embodiment, the diabetes management device 19 further comprises a microphone 7 for entering voice messages for diary keeping purposes. Additionally, dedicated functions of the diabetes management device 19 and/or an insulin pump may be controlled by voice as will be described in more detail below.

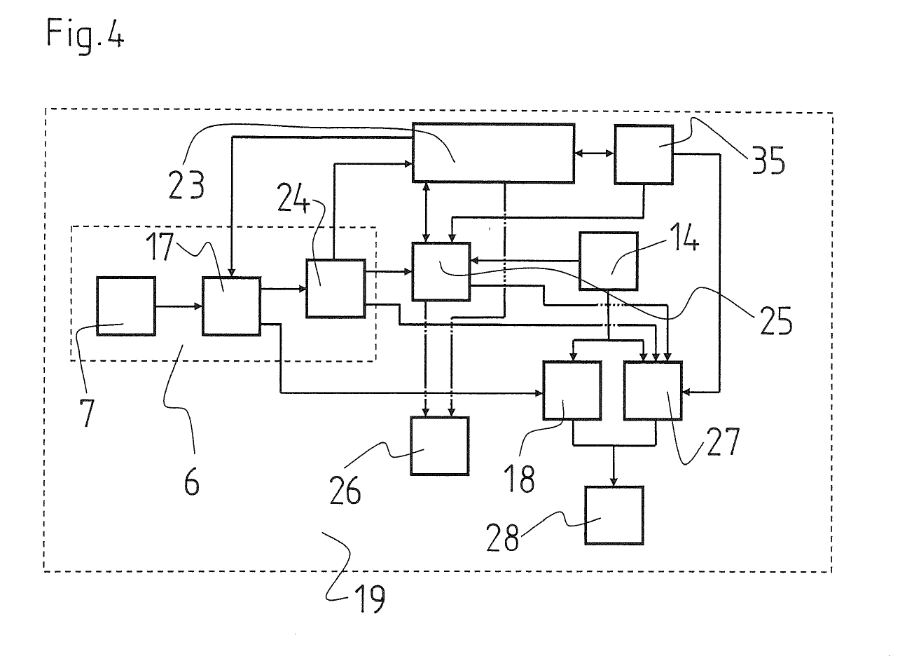

Referring now to FIG. 4, a simplified structural view of the diabetes management device 19 is schematically depicted. In one embodiment, the diabetes management device 19 comprises a user interface 23 comprising the keyboard 21 (FIG. 3), the display 22 (FIG. 3), and non-visual indicators (not shown) such as, for example, a buzzer and/or a pager vibrator.

In another embodiment the diabetes management device 19 comprises a voice recording unit 6 comprising a microphone 7 and a processing unit 17 as described herein. The voice recording unit 6 may be activated at any time via the user interface 23 for entering a voice message. Additionally, the voice recording unit 6 may be automatically activated when a blood glucose measurement is performed with a glucose measurement module 35, which may be disposed within the diabetes management device 19, to record voice information with respect to the measurement and/or a measured value.

The voice recording unit 6 may further comprise a text recognition unit 24 coupled to the processing unit 17 for automated voice-to-text transformation of a selected number of keywords and/or of numeric values. Keywords may be used for simple entries of typical and frequent comments such as “sports,” “hypoglycemia,” “stress,” “dinner,” “snack,” and the like.

The text recognition unit 24 may be operatively coupled to a bolus computation unit 25. The bolus computation unit 25 automatically computes an appropriate insulin bolus dose to be infused by the insulin pump based on the time of day, the size of a meal and/or a recently measured blood glucose value. Bolus computation may be further based upon the therapy history over the last few hours based on therapy data stored in the data memory 27. Exemplary bolus computation units are disclosed in, but not limited to, WO 2006/066926 A1.

A bolus dose computed by the bolus computation unit 25 may be displayed by the display 22, such for confirmation or modification via the keyboard 21. After acknowledgement, a bolus delivery command may then be transmitted to an insulin pump via the pump interface 26.

In one embodiment, the bolus computation unit 25 is coupled with the text recognition unit 24 for receiving spoken data. For example, a person with diabetes may enter a meal size by speaking a numeric value such as an estimated number of grams of carbohydrate, or carbohydrate exchange units which is used as input value for the bolus computation. Additionally, further characterizing information such as protein content, fat content and the relative amounts of liquid to solid components may be entered verbally, stored, and processed. In embodiments where information is stored and/or processed separately, the diabetes management device 19 may indicate the order in which the data should be entered, for example, by displaying text messages indicative of a data entry sequence on a display 22. The patient may enter the data accordingly via voice messages and separate the different data, for example, by pressing a button each time a new data has been entered. Additionally, the patient may enter the data in an arbitrary order and start each entry with a keyword which may be recognized by the text recognition unit 24 for sorting of the data sequence.

A numeric value recognized by the text recognition unit 24 and the insulin bolus amount which is to be infused may be stored in the data memory 27. The voice message may be stored in the voice memory 18 in the same way as further voice notes. Meal information may also be directly entered via the user interface 23. Additionally, the insulin pump may be remotely controlled via the user interface 23.

Even if meal size information is entered via the voice recording unit 6, some embodiments may require data entry to be initiated via the user interface 23 in advance.

Data entered via the voice recording unit 6 and automatically transformed to text by the text recognition unit 24 may be displayed and/or modified via the user interface 23 prior to storage in the data memory 27.

Information stored in both the voice memory 18 and the data memory 27 may be transmitted to an external computer, such as a PC, via a computer interface 28, such as an IR interface or a USB interface. The computer interface 28 may alternatively be integral with the pump interface 26. The computer interface 28 may be further used for configuration of the diabetes management device 19, setting the time and date of the clock circuitry 14, and the like.

The diabetes management device 19 may further comprise a food database (not shown) which is operatively coupled to the bolus computation unit 25. Such a food database stores the absolute or relative carbohydrate amounts such as carbohydrate amount per 100 grams of diverse foods (e.g., fruits, bread, pasta and the like. The food database may also comprise relative sizes (e.g., “small,” “medium,” and “large”) for foods such as pizza, hamburgers, and the like. The text recognition unit 24 may be configured to recognize the meal type and meal size information which is stored in the food database and automatically perform any manual calculations or any searches for information in lists and tables. A combination of a voice recording unit 6, a bolus computation unit 25 and food database enables efficient data entry (e.g., by speaking “pizza small,” “potatoes 150 grams,” and the like).

In further embodiments, the voice recording unit 6 is integrated with the text recognition unit 24 and can be used for controlling the operation of the diabetes management device 19 and/or an insulin pump, and for entering voice notes and meal size information. In another embodiment, the voice recording unit 6 may be used for the entry of voice messages without a text recognition unit 24.

Referring collectively to FIGS. 3 and 4, the diabetes management device 19 may comprise a playback unit for playing back of voice messages stored in the voice memory 18.

For clarity reasons, it is noted that FIG. 4 shows only those components which are of relevance in a framework of the embodiments described herein. Further components, such as a power supply and a controller unit which controls the overall operation at the diabetes management device 19, are not shown but will be apparent to a person skilled in the art. It is further noted that several functional components of the diabetes management device 19 may be realized in an integrated way using known and/or application-specific electronics circuitry.

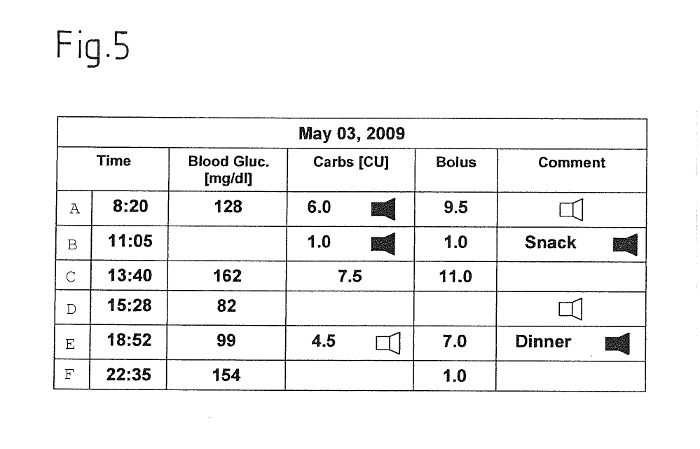

Referring now to FIG. 5, data entered into and stored by the diabetes management device 19 (FIGS. 3 and 4) may be presented in a table-like diary. It is to be understood that FIG. 5 is provided for clarity and is not intended to limit the embodiments described herein to any display format. Dependent on the processing and displaying capabilities of the diabetes management device 19, a large variety of tables, graphs, and mixed presentations may be used. Further data which are not shown in FIG. 5 such as temporary adjustments of an insulin pump may be displayed as well. In the proceeding paragraphs the entries of the table of FIG. 5 will described with reference to line references which correspond to the character in the far left column of the referenced line.

According to the entry in Line A, a blood glucose value of 128 mg/dl was measured at 8:20 and an insulin bolus of 9.5 IU (International Units) was administered for compensating for the intake of 6.0 CU (Carbohydrate Units) of carbohydrates. International Units and Carbohydrate Units are well known in the field of diabetes therapy as measures for insulin amounts and carbohydrate amounts, respectively. The bolus amount may be computed by the bolus computation unit 25 (FIG. 4), based on the entered carbohydrate intake and potentially also the blood glucose value.

Referring still to the entry of Line A, the filled loudspeaker symbol in the “Carbs” cell indicates that the carbohydrate amount was entered via the voice recording unit 6 and transformed into numeric information by the text recognition unit 24. By selecting a cell which shows a loudspeaker symbol such as via the keyboard, the recorded voice information can be played back for verification purposes. Generally, in FIG. 5, a filled loudspeaker symbol indicates a voice message which was successfully processed by the text recognition unit 24.

The loudspeaker symbol in the “Comment” cell of Line A indicates that a further voice message was received, for example a further meal characterization. The loudspeaker symbol is unfilled because the voice message could not be transformed into text by the text recognition unit 24. Generally, in FIG. 5, an unfilled loudspeaker symbol indicates a voice message which was unsuccessfully processed by the text recognition unit 24. By selecting the cell, however, the voice message may be played back.

Line B indicates that the diabetic ate a snack meal at 11:05. Here, the loudspeaker symbol in the “Comment” cell is filled, indicating that the word “Snack” was entered as voice message and automatically was transformed into text by the text recognition unit 24.

Line C comprises no cells with loudspeaker symbol, thus indicating that the diabetic manually entered the carbohydrate information via the keyboard 21 and did not enter a further voice comment.

In Line E, the loudspeaker symbol in the “Carbs” cell is not filled. An unfilled loudspeaker symbol in the “Carbs” cell indicates that a corresponding voice message is present which, however, does not correspond to the numeric carbohydrate information. This may be the case if the text recognition unit 24 could not successfully transform the voice message into text, e.g. due to acoustic distortions, or the diabetic decided to manually modify the value after entry. The entry “Dinner” in the “Comment” cell was entered via the voice recording unit 6 and successfully transformed into text by the text recognition unit 24.

If the diabetes management device 19 is configured to evaluate and/or analyze the data, it may further be configured to actively request the diabetic to retrospectively add further voice comments if the data indicate an unusual and/or exceptional situation that may need further explanation. Typical examples are hypoglycemic or hyperglycemic events, the delivery of a bolus of unusual size and/or time, an unexpectedly raised glucose value after a meal, and the like.

In one embodiment, the diabetes management device 19 is configured to be coupled to further devices, such as an insulin pump. The diabetes management device 19 may request a user such as a diabetic to enter comments on alarms, warnings or errors which are or have been generated by the further devices. For example, the diabetes management device may request a comment upon the occurrence of an occluded infusion cannula or a device error.

Referring again to FIG. 3, the diabetes management device 19 may be handheld and designed to be carried by a user in virtually every situation. Thus, while the diabetes management device 19 incorporates fundamental diary and evaluation functions, the processing, storing and displaying capabilities of the diabetes management device 19 may be limited. In addition, many users use more than one device in parallel, each device generating and storing therapy-related data which are valuable for diary keeping and therapy evaluation purposes.

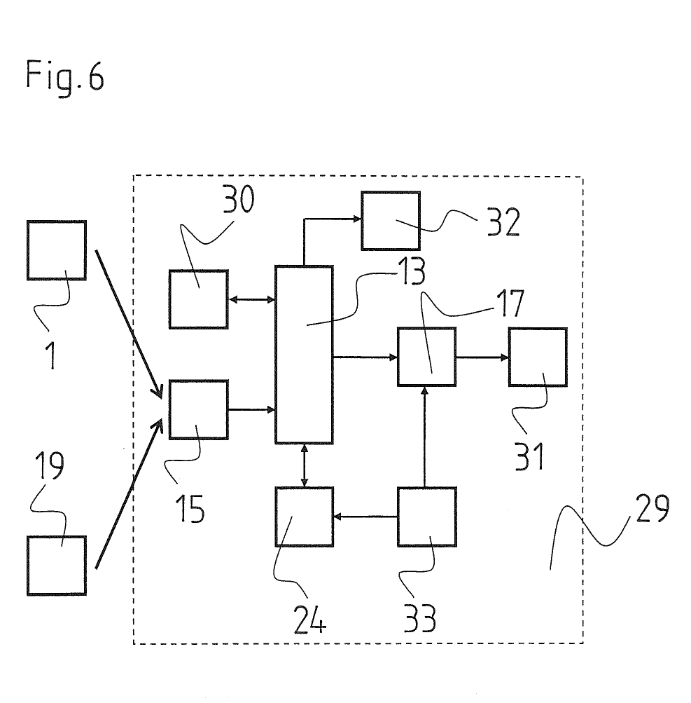

Therefore, the embodiments described herein may be used in combination with an external device 29 to form a system. With reference to FIG. 6, the external device 29 may be a dedicated device or a standard computer, such as a PC and equivalent devices.

In one embodiment, the external device 29 comprises a data interface 15 for coupling to a diary device, a medical device 1, and/or diabetes management device 19 such as, for example, insulin pens, insulin pumps, blood glucose meters and diabetes management devices. The data interface 15 may be, for example, an USB interface, a wired or wireless RS-232 interface, a Bluetooth interface, an RF interface, or the like. The data transmitted to the external device 29 are stored in a memory 13, for example a volatile semiconductor memory. For permanent storage, an additional storage unit 30, such as a hard disk may be provided. A processing unit 17 is operatively coupled to the memory 13 and to an output unit 31, such as a graphic display or computer monitor. The processing unit 17 processes and evaluates the data stored in the memory 13 to generate diary tables, diagrams, charts, statistical evaluations and the like, for presentation via the output unit 31.

The external device 29 may further comprise a text recognition unit 24 and a playback unit 32. These units, in combination, allow automated transformation of voice messages into text even if the diary device or medical device 1 does not include a text recognition unit 24. The capability of the external device 29 to transform voice messages to text may be utilized to supplement the transformation capabilities of a device connected to the data interface 15. For example, the external device may utilize more powerful hardware and/or software to transform voice messages which could not be transformed by a text recognition unit 24 of a diary device, a medical device 1, and/or a diabetes management device 19.

Furthermore, the text recognition unit 24 of the external device 29 may be configured to “learn” new vocabulary by playing a voice message back and subsequently entering the corresponding text via an input unit 33 of the external device 29. The input unit 33 may, for example, comprise a keyboard, a mouse, a touch screen, or the like. The input unit 33 may also be used for the overall controlling of a diary device, a medical device 1, and/or a diabetes management device 19.

After teaching vocabulary into the text recognition unit 24 of the external device, the vocabulary may be transferred to the text recognition unit 24 of a diary device, a medical device 1, and/or a diabetes management device 19 via the data interface 15.

The text recognition unit 24 of the external device 29 may be a dedicated hardware and/or software module or may make use of commercially available text recognition software, such as “Dragon Naturally Speaking” available from Nuance Communications, Inc, Burlington, Mass., USA.

It will be apparent to those skilled in the art that various modifications and variations can be made to the embodiments described herein without departing from the spirit and the scope of the claimed subject matter. Thus it is intended that the specification cover the modifications and variations of the various embodiments described herein provided such modifications and variations come within the scope of the appended claims and their equivalents.

What is claimed is:

1. A method for recording data for keeping a diary, the method comprising:
storing primary data indicative of a measured blood glucose level and/or an administrated dose of insulin in a memory of an electronic diary device;
receiving a spoken voice message with a microphone, wherein the spoken voice message is indicative of supplementary data directly related to the primary data; and
storing the supplementary data in the memory of the electronic diary device, wherein the primary data is stored with a primary time stamp and the supplementary data is stored along with the primary data and the primary time stamp or is stored separately with a supplementary time stamp.

2. The method of claim 1 further comprising:
receiving a further spoken voice message with the microphone, wherein the further spoken voice message is indicative of additional supplementary data that is not directly related to the primary data; and
storing the additional supplementary data in the memory of the electronic diary device with an additional time stamp.

3. The method of claim 1 further comprising:
storing the spoken voice message in the memory of the electronic diary device with the supplementary time stamp or the primary time stamp;
transforming automatically the spoken voice message into a text with the electronic diary device; and
storing the text in the memory of the electronic diary device with the primary data, the primary time stamp or the supplementary time stamp.

4. The method of claim 1 further comprising:
identifying a keyword and/or a numeric value in the spoken voice message with the electronic diary device; and
transforming the keyword and/or the numeric value into a text with the electronic diary device; and
storing the text in the memory of the electronic diary device with the primary data, the primary time stamp or the supplementary time stamp.

5. The method of claim 1 further comprising testing a glucose level in blood of a person to generate the measured blood glucose level, wherein:
the electronic diary device is connected with a means for testing the glucose level in the blood of the person;
the primary data is indicative of the measured blood glucose level; and
the primary data is automatically stored in the memory of the electronic diary device upon the testing of the glucose level in the blood of the person.

6. The method of claim 1 further comprising administering a bolus dose of insulin to a person, wherein:
the electronic diary device is connected with an insulin pen or an insulin pump;
the primary data is indicative of the bolus dose of insulin; and
the primary data is automatically stored in the memory of the electronic diary device upon administering of the bolus dose of insulin to the person.

7. The method of claim 6 wherein the insulin pen or the insulin pump is controlled via the electronic diary device.

8. The method of claim 6 further comprising:
calculating with the electronic diary device a specific amount of insulin to be administered as the bolus dose based at least partially on the spoken voice message; and
initiating the administering of the bolus dose of insulin via the electronic diary device.

9. The method of claim 1 further comprising:
storing malfunction information indicative of a malfunction or a defect of the electronic diary device and/or of a device connected to the electronic diary device in the memory of the electronic diary device with a malfunction time stamp; and
storing explanatory information related to the malfunction information in the memory of the electronic diary device with the malfunction information, the malfunction time stamp or an explanatory time stamp, wherein the explanatory information is an explanatory voice message and/or an explanatory text.

10. A medical device for keeping a diary of a long-term testing of glucose levels in blood of a person and/or of an insulin therapy of the person, the medical device comprising an electronics module comprising:
a memory which stores primary data with a primary time stamp and supplementary data either along with the primary data and the primary time stamp or separately with a supplementary time stamp, the primary data indicative of a measured blood glucose level and/or an administrated dose of insulin and the supplementary data being directly related to the measured blood glucose level and/or the administrated dose of insulin;
a clock circuitry which generates the primary time stamp and the supplementary time stamp; and
a voice recording unit which receives a spoken voice message, wherein the voice message is indicative of the supplementary data.

11. The medical device of claim 10 further comprising a text recognition unit which transforms the spoken voice message into text to be stored in the memory with the primary time stamp or the supplementary time stamp.

12. The medical device of claim 11 wherein the text recognition unit identifies and transforms into the text only keywords and/or numeric values identified in the spoken voice message.

13. The medical device of claim 10 further comprising a means for testing a glucose level in the blood of the person and/or a means for administration of insulin, wherein:
the memory is linked together with the means for testing the glucose level in the blood of the person and/or with the means for administration of insulin; and
upon a testing of the glucose level and/or upon an unscheduled administration of insulin, the primary data is stored automatically in the memory with the primary time stamp or the supplementary time stamp.

14. The medical device of claim 13, wherein the electronics module and the means for testing the glucose level in the blood of the person and/or the means for administration of insulin are each contained in separate housings and are linked with each other via a wireless interface.

15. The medical device of claim 13 further comprising a means for computation of a dose of insulin to be administered based at least partially upon the spoken voice message.

16. The medical device of claim 13 wherein the means for testing the glucose level in the blood of the person and/or the means for administration of insulin are controlled by the electronics module.

17. The medical device of claim 13 wherein the memory stores malfunction information with a malfunction time stamp, the malfunction information indicative of a malfunction or a defect of the electronics module, the means for testing the glucose level in the blood of the person, or the means for administration of insulin.

18. The medical device of claim 17 wherein the memory stores explanatory information related to the malfunction information with the malfunction information, the malfunction time stamp or an explanatory time stamp and the explanatory information is an explanatory voice message and/or an explanatory text.

19. A system for keeping a diary comprising a medical device and an external device, wherein the external device downloads data from the medical device via a wired or wireless interface and the medical device comprises:
a memory which stores primary data together with a primary time stamp and supplementary data together with the primary time stamp or a supplementary time stamp, the primary data indicative of a measured blood glucose level and/or an administrated dose of insulin and the supplementary data directly related to the measured blood glucose level and/or the administrated dose of insulin;
a clock circuitry which generates the primary time stamp and the supplementary time stamp; and
a voice recording unit which receives a spoken voice message, wherein the voice message is indicative of the supplementary data.
